(19) United States
(12) Patent Application Publication
(10) Pub. No.: US 2020/0323677 A1
(43) Pub. Date: Oct. 15, 2020

(54) MANDIBULAR REPOSITIONING DEVICE AND METHODS OF USE THEREOF

(71) Applicant: John R. DROTER, Bowie, MD (US)

(72) Inventor: John R. DROTER, Bowie, MD (US)

(21) Appl. No.: 16/379,716

(22) Filed: Apr. 9, 2019

(51) Int. Cl. A61F 5/56; A61C 7/36; A61C 7/08

(52) U.S. Cl. CPC A61F 5/566; A61F 2005/563; A61C 7/08; A61C 7/36

(57) ABSTRACT

Some embodiments are directed toward a guided airway device for insertion into a user's mouth that includes an upper row of teeth and lower row of teeth, the upper and lower rows of teeth including front teeth. The guided airway device includes a lower appliance that generally conforms to and at least partially covers the user's lower row of teeth, the lower appliance having two ends such that an approximate center point is disposed adjacent the user's front teeth. The guided airway device also includes an upper appliance that generally conforms to and at least partially covers the user's upper row of teeth, the upper appliance having two ends such that an approximate center point is disposed adjacent the user's front teeth. The guided airway device further includes a guide that laterally biases the upper and lower appliances so as to laterally separate the user's upper and lower front teeth.

### BACKGROUND

Obstructive sleep apnea (OSA), a sleep disorder characterized by recurring collapse of the upper airway during sleep, is defined by the occurrence of five or more episodes of complete (apnea) or partial (hypopnea) upper airway obstruction per hour of sleep (Sutherland et al., 2014). OSA, and resultant sleep fragmentation and oxygen desaturation, have been associated with daytime sleepiness, cognitive impairment, type 2 diabetes, cardiovascular disease, stroke, increased risk of motor vehicle accidents and can have significant effects on quality of life. It can affect 4% to 9% of the middle-aged population, and some studies report an incidence as high as 34% in men. Sufferers require an effective and long-term treatment.

A standard treatment for OSA is to pneumatically splint the upper airway during sleep using continuous positive airway pressure (CPAP). Although CPAP is highly efficacious in preventing upper airway collapse, providing a successful outcome in over 95% of users, as many as 50% of patients cannot tolerate the use of CPAP. Hence many patients look toward alternative treatment options, such as the use of a mandibular advancement device (MAD).

### SUMMARY

Sleep disordered breathing (SDB) is a progressive disease that affects both adults and children. Obstructive Sleep Apnea (OSA) is the end stage of the SDB progression. OSA is defined by an Apnea Hypopnea Index (AHI) of 5 or greater. Oxygen desaturation of either 3% or 4% is the criteria for a hypopnea depending on which scoring rules are utilized. There are many people who are “pre” apnea/hypopnea. The majority of diagnostic criteria and screening tools are designed to find obstructive sleep apnea and not the earlier stages. Since it is much easier to treat any disease in its earlier phases, identifying a patient with SDB before they develop OSA is immensely beneficial since therapies can be applied to stop the disease progression.

Disordered breathing is divided into 4 stages. Stage 1 is the presence of predisposing factors that directly or indirectly limit the size of the airway. Predisposing factors of Stage 1 include: Tongue tie, lip tie, bottle fed as infant, dysfunctional swallow, allergies, nasal obstruction, enlarged tonsils, enlarged adenoids, large tongue, mid-face deficient growth, mandibular deficient growth. Stage 2 is the body compensating for deficiencies in the airway thereby maintaining adequate blood oxygen saturation. Stage 2 compensations include: Tongue Bracing, head postured forward, jaw postured forward, mouth breathing. Stage 3 is the airway partially collapses during sleep, with a resultant upper airway resistance. Signs and symptoms of Stage 3 include: Upper Airway Resistance (UAR), 2% drop in oxygen saturation, respiratory effort related arousal (RERA), decrease in deep sleep, decrease in production of growth hormone, weight gain, heart rate fluctuation, snoring or “purring”, cognitive impairment, attention deficit disorder, hyperactivity. Stage 4 is a complete collapse of the airway during sleep with resultant apnea. Signs and symptoms of stage 4 include: 3+% drop in blood oxygen saturation, apnea, cardiovascular damage, elevated blood pressure, gastroesophageal reflux disease, tooth grinding during sleep.

TABLE 1
Disordered Breathing Disease Progression

| Disease Stage 1 | Disease Stage 3 | Disease Stage 3 | Disease Stage 4 |
|---|---|---|---|
| Predisposing | Compensation: | Sleep | Sleep |
| Factors | Airway Maintained | Airway Partial Collapse | Airway Full Collapse |
| Tongue Tie, Lip Tie | Signs: | Signs: | Skins: |
| Bottle Fed as Infant | Tongue Bracing | All of stage 1 and 2 plus . . . | 3+% drop O2 |
| Dysfunctional Swallow | Indents in Tongue | Upper Airway Resistance | Saturation |
| Allergies | Head Postured Forward | 2% Drop O2 Saturation | Apnea |
| Nasal Obstruction | Jaw Postured Forward | RERA- Respiratory Arousals | Cardiovascular |
| Large Tonsil | Sore Masseters | [symbol] Growth Hormone | Damage Elevated BP |
| Large Adenoids | Sore Neck Muscles | Symptoms: | GERD |
| Large Tongue | Mouth Breathing | Heart Rate Fluctuation | Sleep Teeth Grinding |
| Mid-face Deficient | Symptoms: | Snoring or “Purring” | Symptoms: |
| Mandibular Deficient | Facial Ache | Weight Gain | All of stage 2, 3 plus |
| Small Airway | Not Waking Rested | Cognitive Impairment, ADD | Worn Teeth |
| | Daily Fatigue | Hyperactivity | |
| | Neck Soreness | | |

TABLE 2
Disordered Breathing Disease Stage 4
OSA—Obstructive Sleep Apnea
AHI—Apnea Hypopnea Index
Apnea and Hypopnea events per hour
Apnea—Stop airflow for 10 seconds
Hypopnea—<50% airflow or 3+% O2 Desaturation

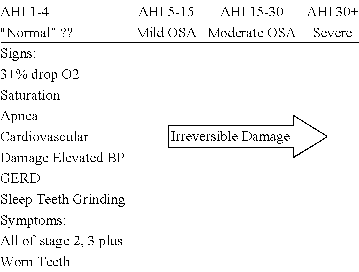

The current clinical practice guideline of the American Academy of Sleep Medicine (AASM) and American Academy of Dental Sleep Medicine (AADSM) recommends that adult patients without OSA, who request treatment of primary snoring, be prescribed oral appliances, rather than no therapy. For adult patients with OSA, both oral appliances and CPAP can significantly reduce the apnea-hypopnea index/respiratory disturbance index/respiratory event index (AHI/RDI/REI) across all levels of OSA severity in adult patients. CPAP remains first-line therapy for the treatment of adult patients with severe OSA; however, it is recommended that patients who are intolerant of CPAP therapy or prefer alternate therapy be provided with an oral appliance, rather than no treatment. A study has shown that there was no significant difference in the percentage of mild OSA patients achieving their target AHI/RDI/REI (<5, <10, >50% reduction) after treatment between OAs and CPAP. MAD use instead of CPAP therapy may prove to be more acceptable for patients with chronically impaired nasal ventilation, frequent travelers who prefer the convenience of a MAD, and for residents of areas where electrical power is not available.

The MAD aims to create a widening of the upper airway configuration by posturing the mandible forward. This action changes the dimensions of the upper airway, including the hypopharynx, the oropharynx, and the nasopharynx, and imaging studies have shown that the upper airway space expands, most notably in the lateral dimension of the velopharyngeal region. It has been also hypothesized that MADs increase muscular tonus by increasing the passive muscle tension in the pharyngeal wall, thereby reducing the vibration of the soft tissues and the turbulent airflow. Assessment of pharyngeal collapsibility during mandibular advancement therapy has also shown a dose-dependent effect in improvement of upper airway closing pressures. When oral appliance therapy is prescribed for an adult patient with OSA, a custom, titratable appliance is recommended.

Although MAD therapy has been shown to be effective in the treatment of OSA, its use may be associated with side effects. Beneficial treatment effects may be reduced by treatment-related side effects, and ultimately the lack of adherence to treatment. Excessive salivation, dry mouth, and pain or discomfort in the supporting teeth, oral mucosa, masticatory muscle, and temporomandibular joint (TMJ) have been reported as temporary side effects during short and medium periods of oral appliance use.

Long-term side effects include occlusal changes without the presence of pain, and skeletal changes. Most of these short to medium term side effects are transient and often treatable. It is purported that by inducing a forward and downward position of the mandible and maintaining it in a non-habitual position during sleep, the harmony of the stomatognathic system is potentially affected, with subsequent development of signs and symptoms of TMD.

The American Academy of Orofacial Pain (AAOP) defines TMD as a collective term for a group of musculoskeletal and neuromuscular conditions which includes several clinical signs and symptoms involving the muscles of mastication, the TMJ, and associated structures. In a recent update, the American Academy of Orofacial Pain divided TMD in two broad categories: TMJ disorders and masticatory muscle disorders. TMD affects 5% to 12% of the population, and typically affects the middle-aged population, occurring more frequently in women.

These disorders are principally characterized by pain in the temporomandibular region or in the muscles of mastication, functional limitations or deviations in mandibular range of motion and TMJ sounds during jaw function. The symptoms most often reported by patients include pain in the face, TMJ, masticatory muscles and pain in the head and ear. The signs are primarily muscle and TMJ tenderness to palpation, limitation and/or incoordination of mandibular movements and joint noises. The presence of TMD may result in poor adherence or even discontinuation of treatment. Hence its early diagnosis and treatment is necessary.

Current methods to advance both condyles out of fossa include push, guide mandible (for example Herbst appliance, SUAD, Somnomed appliance), pull mandible (e.g., EMA appliance, Naval CC), hold mandible (e.g., TAP appliance), guide and hold (keeps mouth closed) (e.g., add elastic to SUAD), pull and hold (keeps mouth closed) (e.g., add elastic to Narval CC). Appliances can be either passive or active. Active: actively hold jaw in that position, the patient can be passive. Jaw can not drift open. Passive: requires the patient's jaw to be closed into the appliance. May patient have jaw tension while sleeping and this keeps jaw in the appliance. Adding elastic from the top to the bottom element can transform a passive appliance into an active one.

Current MADs have upper and lower section that is held on the teeth by engaging undercuts. Most devices cover the all teeth but some only cover sections for teeth. Device material is typically Acrylic, Milled Nylon, Cast Metal.

Most devices actively hold the jaw in the forward position. Some are passive in that the jaw is only positioned forward when the patent is biting. Joining of the upper and lower sections so that the jaw is held forward is achieved by, for example: Hooking onto a bar (TAP appliance); Acrylic Ramps (SomnoMed appliance); Acrylic Ramps with elastics (Twin Block appliance); Elastomerics (Myerson EMA); Sliding Rods (Herbst appliance).

All Current Dental Devices to treat Snoring and Obstructive Sleep Apnea move the mandible forward. Both condyles of the mandible (Jaw Joint) are moved out of their socket and held there by the device. The full weight of the lower jaw and attached muscles is supported by the upper portion of the device. 20% of patients who use current mandibular advancement devices (i.e., mandibular advancement appliances, oral appliance therapy, mandibular anterior repositioning devices, etc.) create a permanent change to their bite and only hit on front teeth.

However, the airway also opens if you move jaw laterally. One condyle is advanced out of the fossa while the other one remains seated in the fossa. Less than half the force is required to tip the lower jaw than advance it.

All current devices which advanced both condyles forward out of the fossa, can be modified to advance only one condyle, thus moving the jaw laterally. If the jaw is moved laterally (sideways), the amount of force is significantly less. This is similar to the amount of force d needed to tip a file cabinet is significantly less than force need to lift it. Only one condyle is moved out of the socket.

An exemplary lateral airway assist device (LAAD) opens the airway by changing the dimensions of the upper airway, including the hypopharynx, the oropharynx, and the nasopharynx. The LAAD opens the airway laterally at only one side only, which is enough to prevent obstruction.

Additionally, side effects with the LAAD appear to be much less, possible due to the decrease in forces, and forces in a different direction. Bruxing (grinding) of teeth associated with Obstructive Sleep Apnea may also be treated more effectively with the LAAD.

During patient use, MADs are broken due to the patient attempting to move their jaw sideways during sleep. With the LAAD, the jaw is already sideways.

Diseases can also be treated with LAAD including: Mild to Moderate Obstructive Sleep Apnea; High Blood pressure caused by Obstructive Sleep Apnea; Upper Airway Resistance Syndrome; Snoring; Sleep Bruxing.

Side effects that can be lessened with LAAD use include: TMJ pain; Muscle pain; Sore teeth; Loose teeth; permeant changes to the Occlusion (bite): Posterior open bite; Skeletal Changes, maxilla distalized; Bruxing wear on Device.

### BRIEF DESCRIPTION OF THE DRAWINGS

The disclosed subject matter of the present application will now be described in more detail with reference to exemplary embodiments of the apparatus and method, given by way of example, and with reference to the accompanying drawings, in which:

FIG. 1 is a perspective view of a first embodiment of an exemplary mandibular advancement device in accordance with the present invention.

FIG. 2 is a perspective view of the first embodiment of an exemplary mandibular advancement device with the upper and lower appliances shown disconnected.

FIG. 3 is a perspective view of a patient's upper and lower arches of teeth.

FIG. 4 is a perspective view of a first embodiment of an exemplary mandibular advancement device in accordance with the present invention with the patient's mouth in a closed position.

FIG. 5A is a perspective view of the first embodiment of an exemplary mandibular advancement device in accordance with the present invention with the patient's mouth in a closed position.

FIG. 5B is a perspective view of a side of the first embodiment of an exemplary mandibular advancement device shown in FIG. 5A.

FIG. 6A is a perspective view of the first embodiment of an exemplary mandibular advancement device fitted to adjust a patient's jaw in an alternate direction.

FIG. 6B is another perspective view of the first embodiment of an exemplary mandibular advancement device fitted to adjust a patient's jaw in an alternate direction.

FIG. 7A is a perspective view of another exemplary mandibular advancement device fitted to a patient's mouth.

FIG. 7B is another perspective view of the exemplary mandibular advancement device shown in FIG. 7A fitted to a patient's mouth.

FIG. 7C is another perspective view of the exemplary mandibular advancement device shown in FIG. 7A fitted to a patient's mouth.

FIG. 7D is another perspective view of the exemplary mandibular advancement device shown in FIG. 7A fitted to a patient's mouth.

FIG. 7E is another perspective view of the exemplary mandibular advancement device shown in FIG. 7A fitted to a patient's mouth.

FIG. 7F is another perspective view of the exemplary mandibular advancement device shown in FIG. 7A fitted to a patient's mouth.

FIG. 8A is a schematic cross-sectional representation of a patient's airway under normal conditions.

FIG. 8B is a schematic cross-sectional representation of a patient's airway under upper airway resistance.

FIG. 8C is a schematic cross-sectional representation of a patient's airway under obstructed apnea.

FIG. 8D is a schematic cross-sectional representation of a patient's airway with exemplary mandibular advancement device in place.

FIG. 9 is a perspective view of a variation of the mandibular advancement device.

FIG. 10 is a perspective view of upper and lower appliances of another embodiment of an exemplary mandibular advancement device in accordance with the present invention.

FIG. 11A is a perspective view of the an appliance of the exemplary mandibular advancement device.

FIG. 11B is a perspective view of another appliance of the exemplary mandibular advancement device.

FIG. 11C is a perspective view of another appliance of the exemplary mandibular advancement device.

FIG. 12A is a schematic cross-sectional representation of a patient's jaw in a natural state.

FIG. 12B is a schematic cross-sectional representation of a patient's jaw in a displaced state.

FIG. 13A is a perspective view of another embodiment of an exemplary mandibular advancement device in accordance with the present invention.

FIG. 13B is a side perspective view of another embodiment of an exemplary mandibular advancement device in accordance with the present invention.

FIG. 14A is a perspective view of another embodiment of an exemplary mandibular advancement device.

FIG. 14B is a perspective view of the embodiment of an exemplary mandibular advancement device of FIG. 14A.

FIG. 15A is a perspective view of another embodiment of an exemplary mandibular advancement device in accordance with the present invention.

FIG. 15B is a side perspective view of another embodiment of an exemplary mandibular advancement device in accordance with the present invention.

FIG. 16 is a perspective view of an upper appliance of an embodiment of an exemplary mandibular advancement device.

FIG. 17A is a perspective view of another embodiment of an exemplary mandibular advancement device.

FIG. 17B is an enlarged perspective view of the embodiment of an exemplary mandibular advancement device of FIG. 17A.

FIG. 18 is a front perspective view of another embodiment of an exemplary mandibular advancement device in accordance with the present invention with the patient's mouth in a closed position.

FIG. 19 is a front perspective view of the embodiment of an exemplary mandibular advancement device of FIG. 18 with the patient's mouth in an opened position.

FIG. 20 is a perspective view of an upper appliance of an embodiment of an exemplary mandibular advancement device.

FIG. 21 is a perspective view of a lower appliance of an embodiment of an exemplary mandibular advancement device.

FIG. 22 is a rear perspective view of an exemplary mandibular advancement device

FIG. 23 is a block diagram showing a method in accordance with the disclosed subject matter.

### DETAILED DESCRIPTION OF EXEMPLARY EMBODIMENTS

A few inventive aspects of the disclosed embodiments are explained in detail below with reference to the various figures. Exemplary embodiments are described to illustrate the disclosed subject matter, not to limit its scope, which is defined by the claims. Those of ordinary skill in the art will recognize a number of equivalent variations of the various features provided in the description that follows.

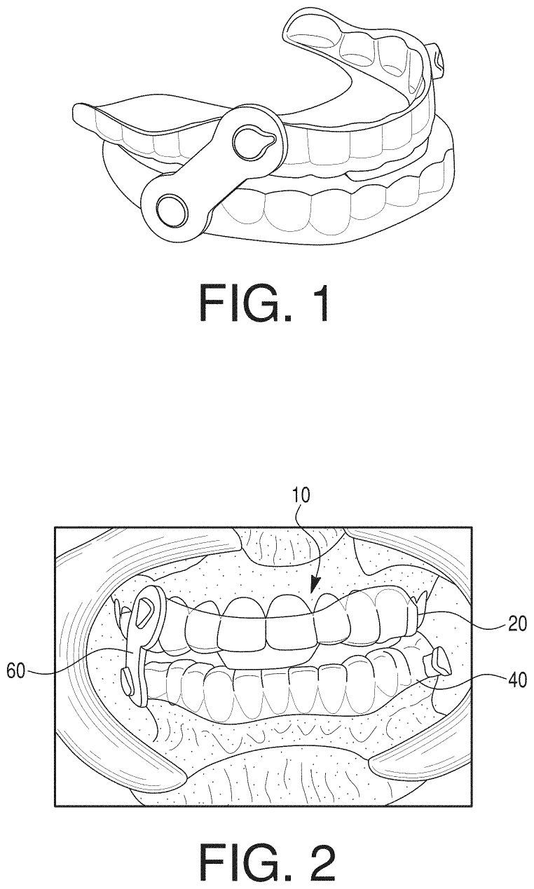

FIG. 1 is a perspective view of a first embodiment of an exemplary mandibular advancement device in accordance with the present invention. In the first embodiment, a mandibular advancement device 10 includes an upper appliance 30 and a lower appliance 60 to fit over a patient's upper arches of teeth 12 and lower arches of teeth 14, respectively. The upper and lower appliances 30, 60 are intended to be worn together by a patient.

The upper and lower appliances 30, 60 are molded and form-fitted to a particular patient's upper and lower arches of teeth 12, 14 so as to be unique to that patient. The upper and lower appliances 30, 60 can be created using 3D scans or molds of the patient's upper and lower arches of teeth 12, 14, or any other scan or impression method.

In the first embodiment, the upper and lower appliances 30, 60 are connected and held together by a band 90. The band 90 is elastic and is anchored to knobs 40, 70 on the upper and lower appliances 30, 60, respectively. The band 90 allows some movement between the upper and lower appliances 30, 60 within the elastic limits of the band 90 while limiting extreme movement therebetween. The knob 40 on the upper appliance 30 can be positioned forward of the knob 70 on the lower appliance 60 such that the knobs 40, 70 are staggered. Alternatively, the knob 70 on the lower appliance 60 can be positioned forward of the knob 40 on the upper appliance 30. The knobs 40, 70 can have integrated hooks to keep the band 90 from slipping or sliding off or out of position. The knobs 40, 70 can be connected or adhered to the upper and lower appliances 30, 60, respectively, or be formed integrally therewith.

The band 90 is formed to be elastomeric so as to urge translational movement between the upper and lower appliances 30, 60, and thereby move the patient's upper and lower arches of teeth 12, 14 correspondingly. The translational movement of the upper and lower appliances 30, 60, and the patient's upper and lower arches of teeth 12, 14 can be described as lateral movement, i.e., right and left side-to-side displacement. The translational movement of the patient's upper and lower arches of teeth 12, 14 is further described below as it relates to opening the patient's airway to alleviate snoring and/or sleep apnea.

The mandibular advancement device 10 of the first embodiment also includes a post 80 on the upper appliance 30. The post 80 is a protrusion can be connected or adhered to the upper appliance 30, or be formed integrally therewith. The post 80 serves to abut a contact surface 62 on the lower appliance 60 and thereby maintain separation space between a remainder of the upper and lower appliances 30, 60. Thus, the separation space between a remainder of the upper and lower appliances 30, 60 ensures corresponding separation space between the patient's upper and lower arches of teeth 12, 14. This separation space prevents the patient from clamping the upper and lower arches of teeth 12, 14 together, thereby allowing the translational movement of the upper and lower arches of teeth 12, 14 laterally. The post 80 and contact surface 62 also serve to limit contact between the upper and lower appliances 30, 60, thereby preventing any damage thereto.

FIG. 2 is a perspective view of the first embodiment of an exemplary mandibular advancement device with the upper and lower appliances shown disconnected. As can be seen in FIG. 1, the mandibular device may include a tab 43 on the lower appliance. The tab 43 may act as a further assistance to separation space and lateral movement.

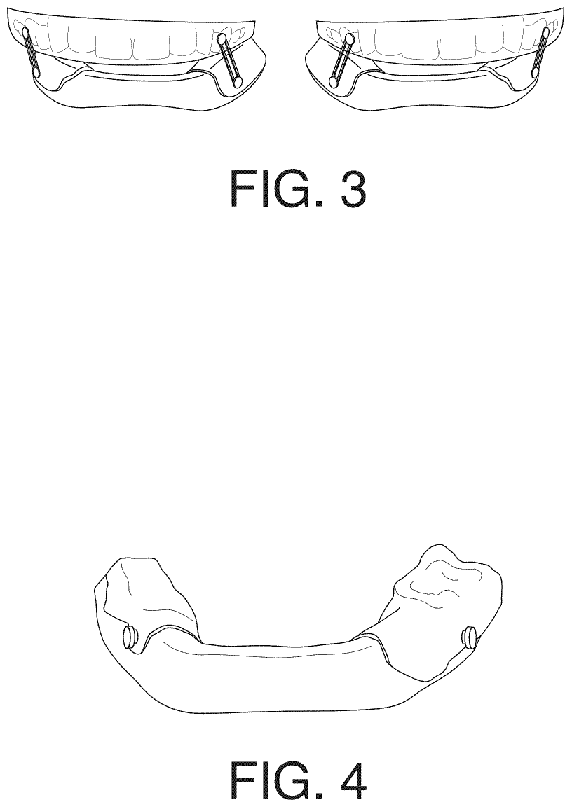

FIG. 3 is a perspective view of a patient's upper and lower arches of teeth. As can be seen in FIG. 3, the patient's upper and lower arches of teeth, 12,14 respectively, are clinched together. Thus, there is no separation space between the arches of teeth 12, 14.

FIG. 4 is a perspective view of a first embodiment of an exemplary mandibular advancement device in accordance with the present invention with the patient's mouth in a closed position. As can be seen in FIG. 2, a mouth is in a closed position and experiencing separation space and lateral movement due to the mandibular advancement device 10.

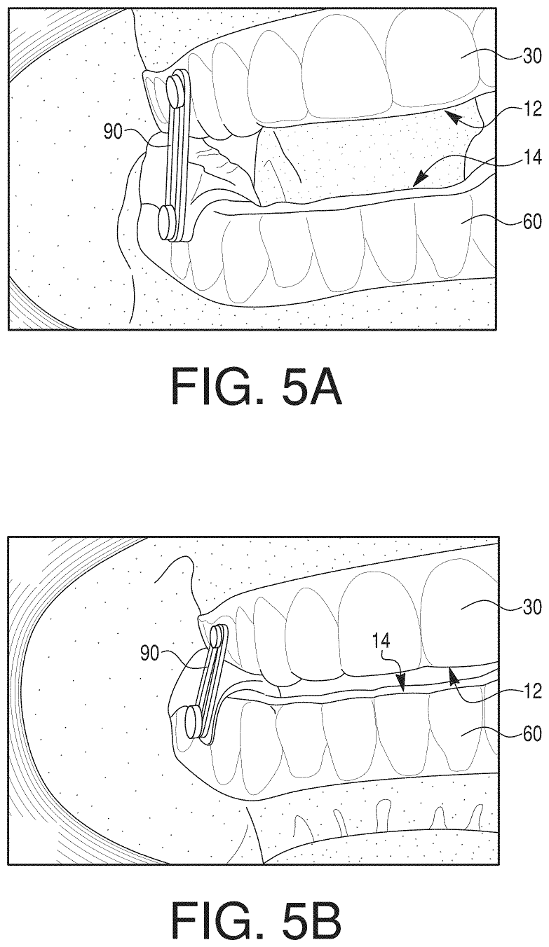

FIG. 5A is a perspective view of the first embodiment of an exemplary mandibular advancement device in accordance with the present invention with the patient's mouth in a closed position. As can be seen in FIG. 4, a post 80 and a band 90 are causing lateral movement with various degrees of separation space between the patient's upper and lower arches of teeth, 12,4 respectively.

FIG. 5B is a perspective view of a side of the first embodiment of an exemplary mandibular advancement device shown in FIG. 5A. As described in FIG. 5A, the mandibular advancement device 10 is causing lateral movement.

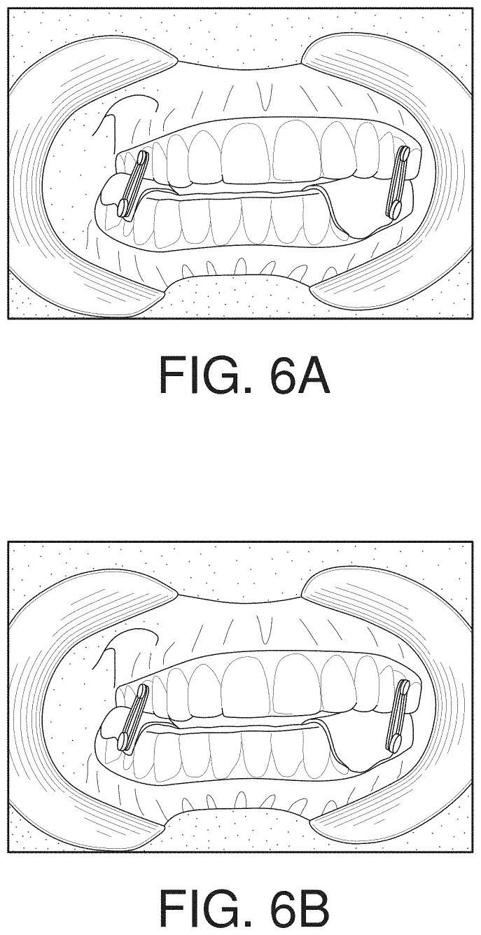

FIG. 6 is a perspective view of the first embodiment of an exemplary mandibular advancement device fitted to adjust a patient's jaw in an alternate direction. As shown in FIG. 5A, the band 90 may be place on multiple positions and orientations of the upper and lower appliances 30, 60.

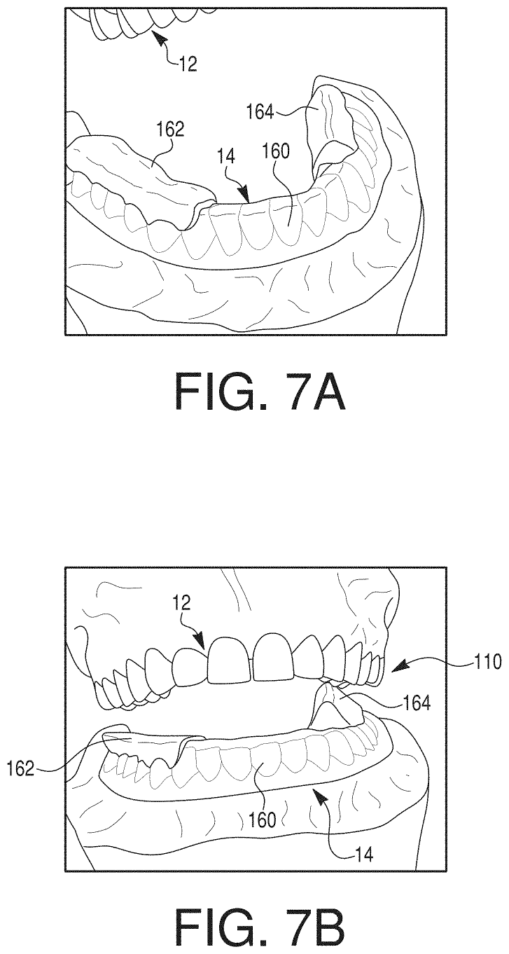

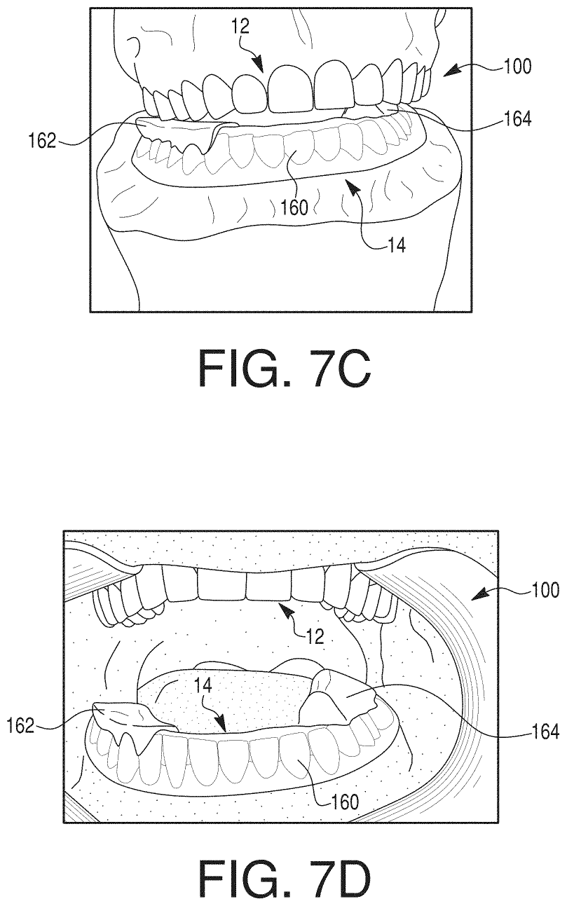

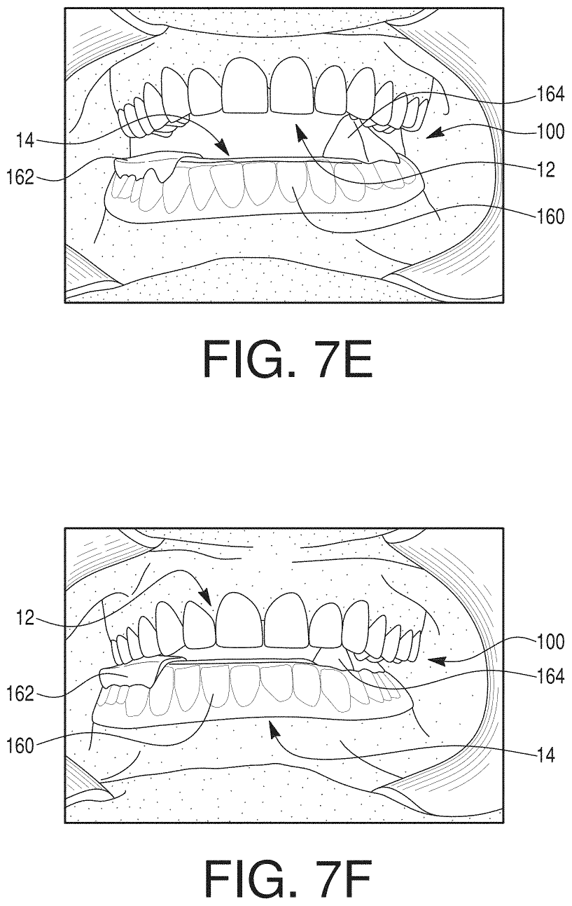

FIG. 7 is a perspective view of the exemplary mandibular advancement device shown in FIG. 1 fitted to a patient's mouth. As can be seen in FIG. 7, the knobs 40, 70 have respective hook parts 42, 72. The band 90 has loop parts 92, 94 with holes that are looped over and onto the knobs 40, 70, respectively, to secure the band 90 in place onto the upper and lower appliances 30, 60. The loop parts 92, 94 must be slid over the hook parts 42, 72 of the respective knobs 40, 70, and once secured, the hook parts 42, 72 ensure accidental displacement or unlooping of the loop parts 92, 94 does not occur.

The band 9 interacts with the upper and lower appliances 30, 60 to urge or pull the lower appliance 30 and patient's lower arch of teeth 14 laterally to one side, either the right or left depending on the disposition and configuration of the band 90 (right or left side connection). The patient's upper arch of teeth 12 anchor the mandibular advancement device 10 via the upper appliance 30, while the lower appliance 60 and patient's lower arch of teeth are displaced laterally by the force of the band 90.

The band 90 can have certain elastomeric characteristics and ratings in accordance with the specifications suitable for a particular patient. The band 90 can be changed or replaced with another band of differing characteristics should the patient's needs require an elastic force of a greater or lesser intensity. The band 90 may have any shape used in the art. The band 90 may have a “link-joint” shape.

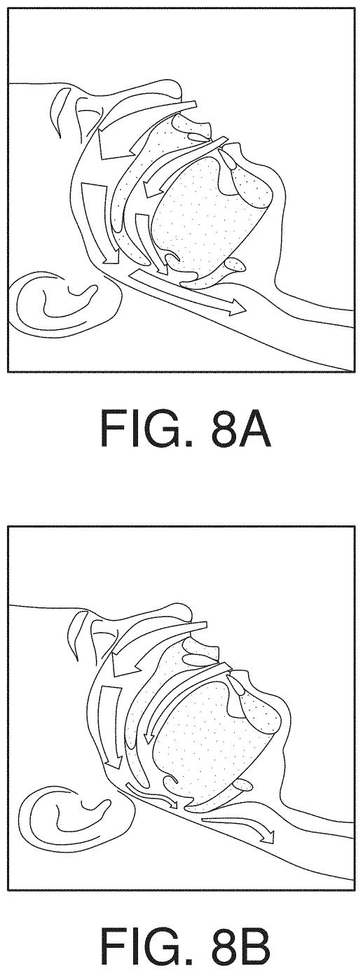

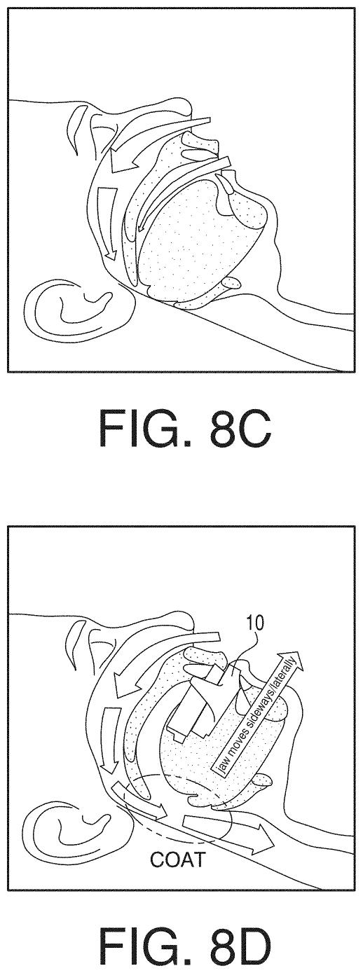

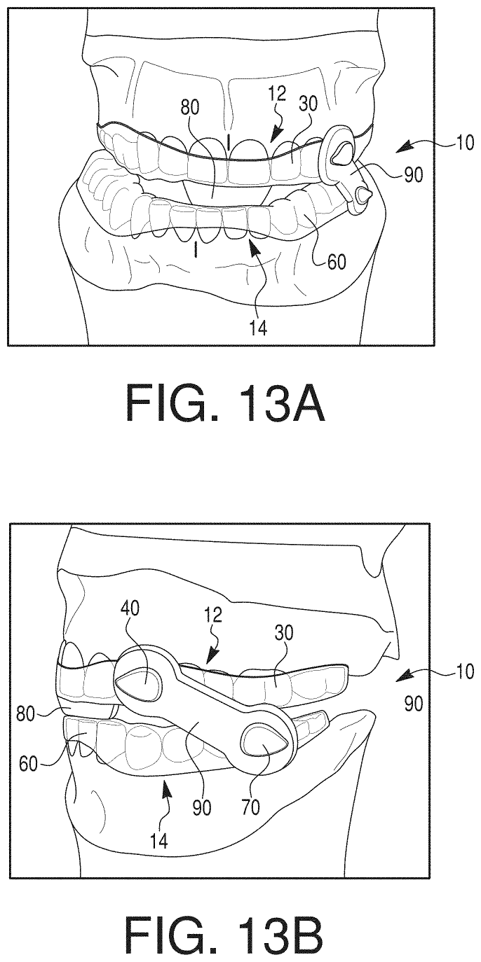

FIG. 8A is a perspective view of an exemplary mandibular advancement device as shown in FIG. 13A. As can be seen in FIG. 8A, a band 90 and a second band 96 are attached to the upper appliance 40 and the lower appliance 60. Band 90 and the second band 96 work in conjunction to provide translational movement between the upper and lower appliances 30, 60, and thereby move the patient's upper and lower arches of teeth 12, 14 correspondingly. Thus, separation space is created between the patient's upper and lower arches of teeth 12, 14. In conjunction the bands 90, 96 can be stretched at different elastomeric orientations. Bands 90, 96 can be spaced towards each other or spaced away from each other depending on the patient's upper and lower dental impression. Another embodiment might feature band 96 creating translation movement and band 90 being used as a preventative measure. Regardless, a multitude of elastic bands may be used.

FIG. 8B is a perspective view of an exemplary mandibular advancement device as shown in FIG. 8A. As can be seen in FIG. 8B, a band 90 and a second band 96 can be used in conjunction to create various types of separation space.

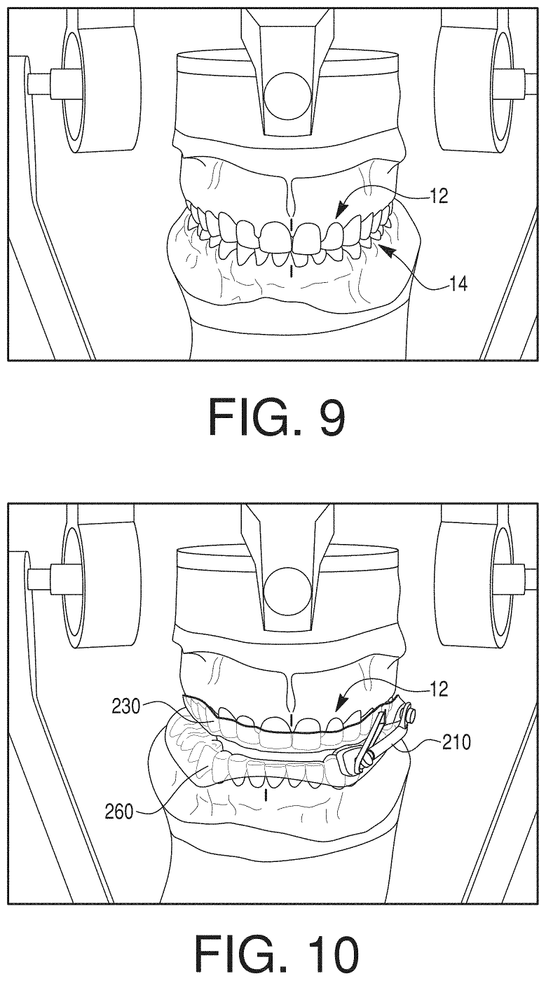

FIG. 9A is a partial perspective view of a variation of the mandibular advancement device shown in FIG. 7 with the patient's mouth in an opened position. As can be seen in FIG. 9A, a band 90 is attached to the knob 40, hook part 42 of the upper appliance 30 and the knob 70, hook part 72 of the lower appliance 60. The band 90 may have a ring shape. The band 90 may be made from any elastic material suitable for dental use.

FIG. 9B is a partial perspective view of a variation of the mandibular advancement device shown in FIG. 7 with the patient's mouth in a closed position. As described above in FIG. 9A, FIG. 9B depicts the lateral displacement that is induced on a patient by the mandibular advancement device 10. The mandibular advancement device creates a separation space. Thus, the separation space between a remainder of the upper and lower appliances 30, 60 ensures corresponding separation space between the patient's upper and lower arches of teeth 12, 14.

FIG. 10 is a perspective view of upper and lower appliances of a second embodiment of an exemplary mandibular advancement device in accordance with the present invention. The second embodiment of the mandibular advancement device 110 shown in FIG. 10 includes upper and lower appliances 130, 160 intended to fit to a patient's upper and lower arches of teeth 12, 14 similarly to the upper and lower appliances 30, 60 described above.

The upper appliance includes a ramp 132 formed thereon, and the lower appliance includes ramps 162, 164 formed thereon. The ramps 132, 162, 164 are protrusions that can be connected or adhered to the upper and lower appliances 30, 60, respectively, or be formed integrally therewith. The ramps may also be configured as post. The ramps 132, 162, 164 serve to abut corresponding and facing contact surfaces 166, 134, 136 on the lower and upper appliances 160, 130, respectively. Specifically, the ramp 132 abuts the contact surface 166, the ramp 162 abuts the contact surface 134, and the ramp 164 abuts the contact surface 136. The ramps 132, 162, 164 and corresponding contact surfaces 166, 134, 136 guide the upper and lower appliances 130, 160 apart to achieve translational movement therebetween, and corresponding movement between the patient's upper and lower arches of teeth 12, 14. Particularly, the lower appliance 160 is moved laterally to either a left or right side of the patient's mouth.

Additionally, the ramps 132, 162, 164 and corresponding contact surfaces 166, 134, 136 maintain separation space between a remainder of the upper and lower appliances 130, 160. Thus, the separation space between a remainder of the upper and lower appliances 130, 160 ensures corresponding separation space between the patient's upper and lower arches of teeth 12, 14. This separation space prevents the patient from clamping the upper and lower arches of teeth 12, 14 together, thereby allowing the translational movement of the upper and lower arches of teeth 12, 14 laterally. The ramps 132, 162, 164 and corresponding contact surfaces 166, 134, 136 also serve to limit contact between the upper and lower appliances 130, 160, thereby preventing any damage thereto.

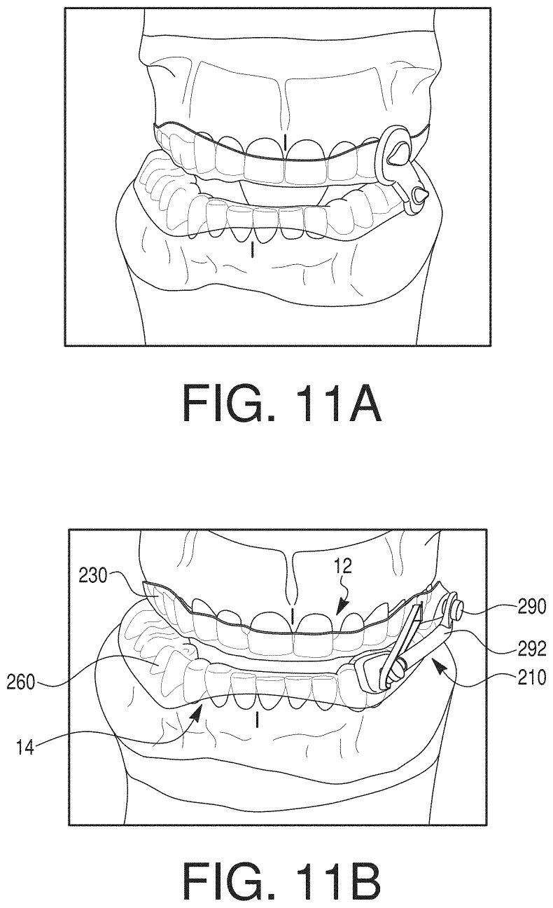

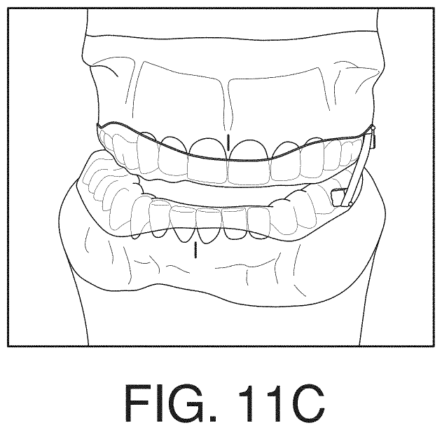

FIG. 11 is a perspective view of the lower appliance of the exemplary mandibular advancement device shown in FIG. 10. As can be seen in FIG. 11, the lower appliance 160 has ramps 162, 164. The ramps 162, 164 have knobs 168, 170 attached thereto. Knobs 168, 170 feature hooks 172, 174.

The ramps 162, 164 are form-fitted about the front (away from patient), back (towards patient), and top (away from patient's bottom jaw line) of the patient's lower arches of teeth 14. Ramps 162, 164 are form-fitted to a shape that features various slope angles 176, 178 about the patient's lower arches of teeth 14. The slope angles 176, 178 are determined during a dental patient assessment. Thus, the ramps 162 and 164 may have the same slope angle 176, 178 or have different slope angles 176, 178 from each other depending on the patient's upper and lower dental impressions. The ramps 162, 164 may be made from any material suitable for dental uses, such as a hard acrylic material, or methylmethacrylate or a polycarbonate resin thermoplastic

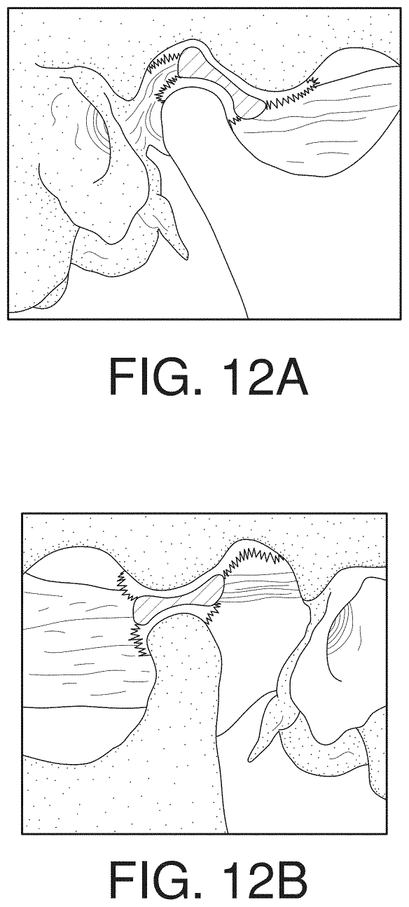

FIG. 12A is a perspective view of a second embodiment of an exemplary mandibular advancement device in accordance with the present invention with the patient's mouth in an opened position. As described above in FIG. 11, ramps 162, 164 are form-fitted about the lower appliance 160. The lower appliance is fitted on the patient's lower arches of teeth. The ramps, 162, 164 may be formed

FIG. 12B is a perspective view of the second embodiment of the mandibular advancement device shown in FIG. 12A with the patient's mouth in an intermediate position. As can be seen in FIG. 12B, an embodiment of the mandibular advancement device 110 may occur with just the lower appliance.

FIG. 12C is a perspective view of the second embodiment of the mandibular advancement device shown in FIG. 7A with the patient's mouth in a closed position. As can be seen in FIG. 12C, the ramps 162, 164 of lower appliance 160 and the patient's upper arches of teeth may still create separation space and lateral movement.

FIG. 12D is a perspective view of a second embodiment of an exemplary mandibular advancement device in accordance with the present invention with the patient's mouth in an opened position. As shown in FIG. 12D, the ramps 162,164 on a lower appliance 160 do not need to have the same dimensions. In an embodiment a excessively protruded ramp 165. Additionally, this possible embodiment may function with just the patient's upper arches of teeth and the lower appliance 160. Another embodiment may have an upper appliance 130 being applied to the patient's lower arches of teeth 14 to cause separation space and later space. Such an embodiment would feature ramp 162, 164, or 165 on the upper appliance 130.

FIG. 12E is a perspective view of the second embodiment of the mandibular advancement device shown in FIG. 12D with the patient's mouth in an intermediate position. FIG. 12E is different mouth position of the device described above in FIG. 12D.

FIG. 12F is a perspective view of the second embodiment of the mandibular advancement device shown in FIG. 12D with the patient's mouth in a closed position. As shown in FIG. 12F, the patient's upper arches of teeth 12 may bite over the excessively protruded ramp 165 and bite directly onto ramp 162. This embodiment is an alternative way of creating lateral movement. The excessively protruded ramp 165 may push behind (direction toward patient) the patent's upper arches of teeth 12 to create lateral movement.

FIG. 13 is a perspective view of a fourth embodiment of an exemplary mandibular advancement device in accordance with the present invention. As shown above in FIG. 19A, in some embodiments the rod 292 may be used in conjunction with the band 290 and post 280 to cause lateral movement and separation space between the upper and lower appliances 230, 260.

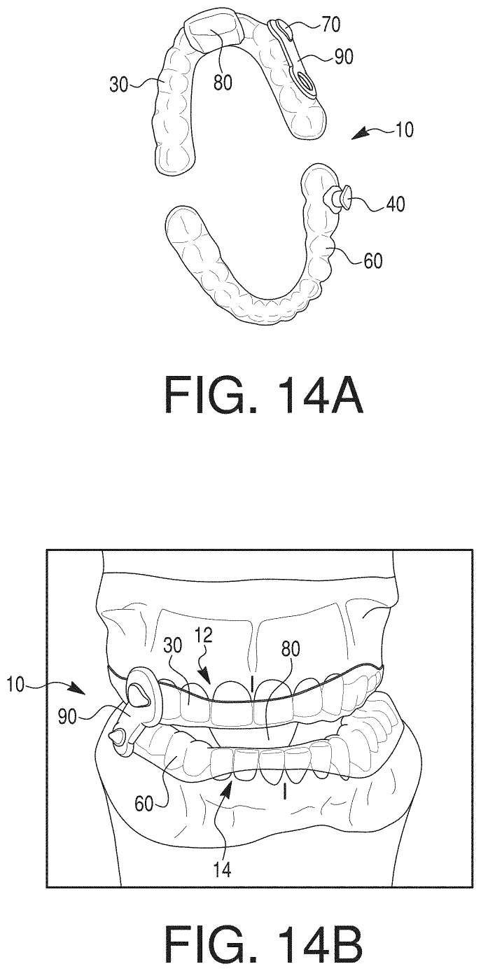

FIG. 14 is an enlarged perspective view of the fourth embodiment of an exemplary mandibular advancement with a patient's mouth shown open. As shown above in FIG. 13, in some embodiments a ramp 250 may be used in conjunction with the rod 292, band 290, and post 280 to cause lateral movement and separation space between the upper and lower appliances 230, 260.

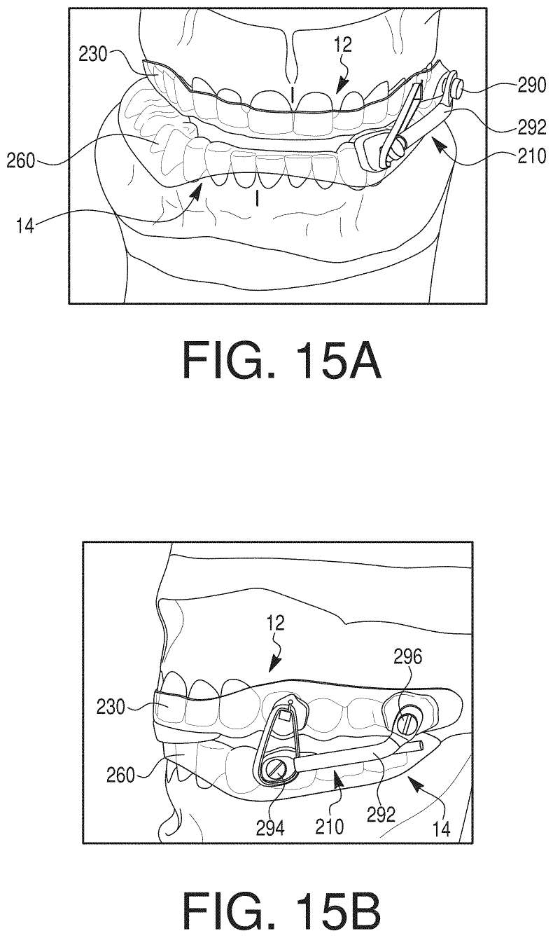

FIG. 15 is a perspective view of an upper appliance of the fourth embodiment of an exemplary mandibular advancement device. In the case of FIG. 25, one embodiment of the upper appliance 330 has a post 380 and knob 340

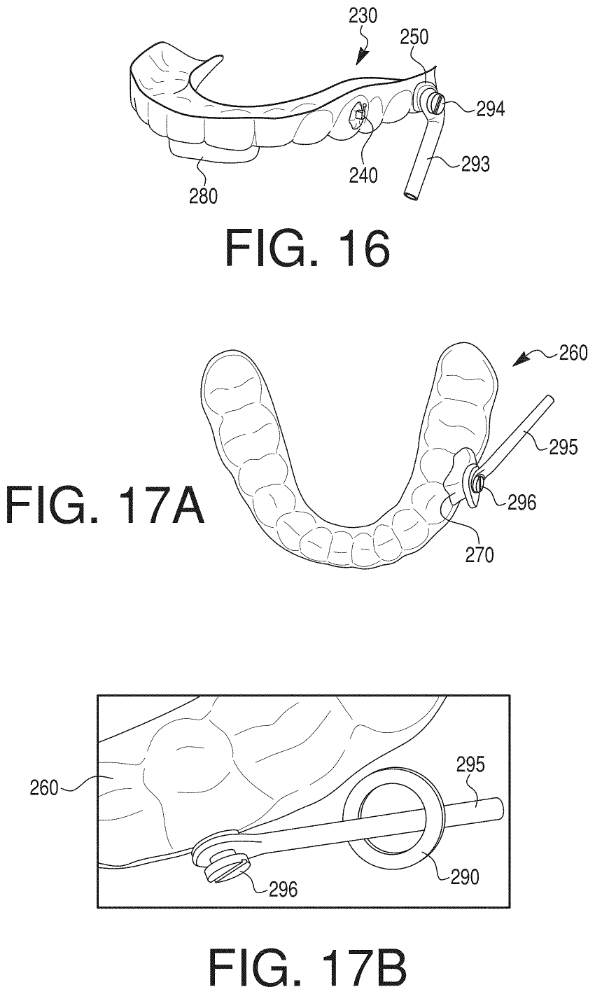

FIG. 16 is a perspective view of a lower appliance of the fourth embodiment of an exemplary mandibular advancement device. In the case of FIG. 16, one embodiment of the lower appliance has a first side ramp 362 and a knob 370.

FIG. 17 is a perspective view of a third embodiment of an exemplary mandibular advancement device in accordance with the present invention with the patient's mouth in a closed position. As seen in FIG. 17 sliding rod 292 is fixed to the upper and lower appliance 230,260. Additionally, a post 280 and band 290 are used in conjunction with the rod 292 to produce separation space and lateral movement between the arches of teeth 12, 14.

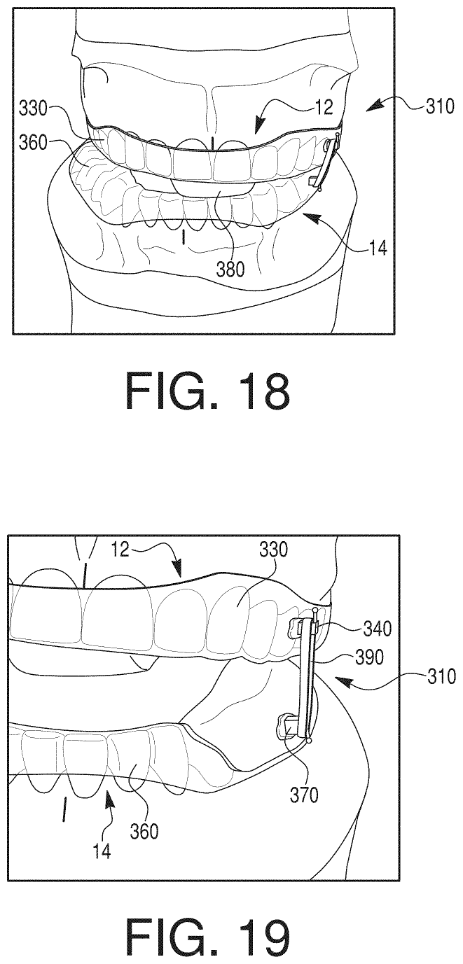

FIG. 18A is a perspective view of a third embodiment of an exemplary mandibular advancement device in accordance with the present invention with the patient's mouth in a closed position. As can be seen in FIG. 18A, a post 280 and band 290 are used in conjunction with the rod 292 to produce separation space and lateral movement between the arches of teeth 12, 14. In this embodiment, the rod 292 and band 290 are mounted on the same places located along the upper and lower appliances 230, 260. In other embodiments, the rod 292 and band 290 are mounted in different locations along the upper and lower appliances 230, 260.

FIG. 18B is a perspective view of a second embodiment of an exemplary mandibular advancement device in accordance with the present invention with the patient's mouth in a closed position. In the case of FIG. 18B, a post 280 located on the upper appliance 230 contacts the lower appliance 260 and created separation space and lateral movement. In some embodiments, only a post is used.

FIG. 19A is a perspective view of the third embodiment of an exemplary mandibular advancement device in accordance with the present invention with the patient's mouth in a closed position. As shown in FIG. 19A, rod 292 may be oriented in a way in which the rod 292 protrudes away from (direction away from the patient) the band 290.

FIG. 19B is a perspective view of a side of the third embodiment of an exemplary mandibular advancement device shown in FIG. 19A. In the case of FIG. 19B, the rod 292 may have a joint 294 and 296. The joints 294, 296 may be permanently fixed and rotate about knobs 250,270 on the upper and lower appliances 230, 260. The band 290 may looped about a hook part 42 and knob 250. Any dental fasteners used in the art may be used to fix the joints 294, 296—such as mini screws and the like. The rod may consist of any material used in the art like noble metals or base-metal allows.

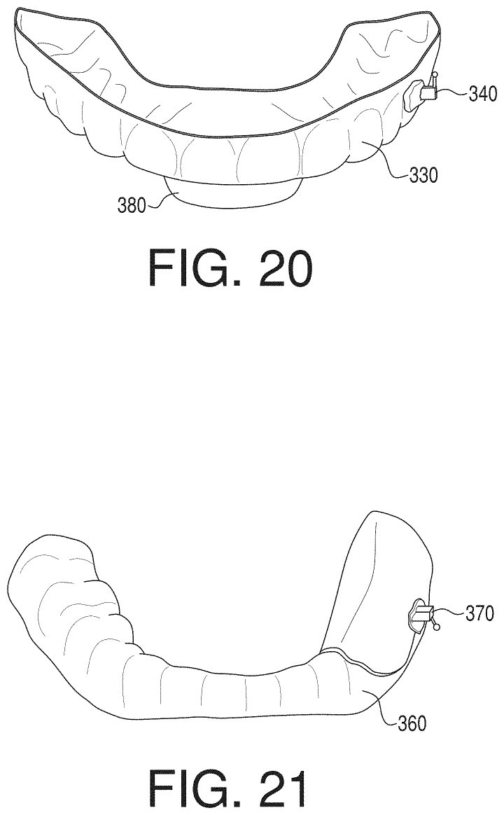

FIG. 20 is a perspective view of an upper appliance of the third embodiment of an exemplary mandibular advancement device. As shown in FIG. 20, the upper appliance 230 of an embodiment may have the rod 292 and its joint 296 permanently fixed and rotate about the knob 270. The outer tube 293 of the rod 292 is permanently fixed about the knob 270.

FIG. 21A is a perspective view of a lower appliance of the third embodiment of an exemplary mandibular advancement device. In the case of FIG. 21A, the lower appliance 260 of an embodiment may have the inner tube 295 of the rod 292 is permanently fixed and rotate about the knob 250. When this embodiment of the mandibular advancement device 210 is in use, the inner tube 295 may be situated inside the outer tube 293 to form the rod 292. The inner tube 295 may be fixed into the outer tube 293 through a force fit or other means in the art for fastening.

FIG. 21B is an enlarged perspective view of the lower appliance of the third embodiment of an exemplary mandibular advancement device shown in FIG. 21A. In the case of FIG. 21B, when no forces are exerted—the band 290 may have a circular with the inner tube 295 running through the middle of the band 290.

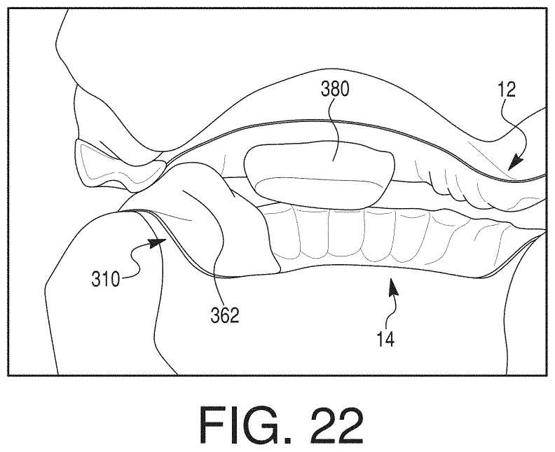

FIG. 22A is a schematic cross-sectional representation of a patient's airway under normal conditions. As shown in FIG. 22A, a patient's airway configuration features air movement through the hypopharynx, oropharynx, and the nasopharynx.

FIG. 22B is a schematic cross-sectional representation of a patient's airway under upper airway resistance. Resistance in a patient's upper airway is occurs due to the dimensions of a patient's hypopharynx, oropharynx, and the nasopharynx changing. As can be seen in FIG. 22B, there is still air flowing through the patient's upper airway.

FIG. 22C is a schematic cross-sectional representation of a patient's airway under obstructed apnea. As can be seen in FIG. 22C, the patient's upper airway is blocked and there is not airway flowing past the naval passage.

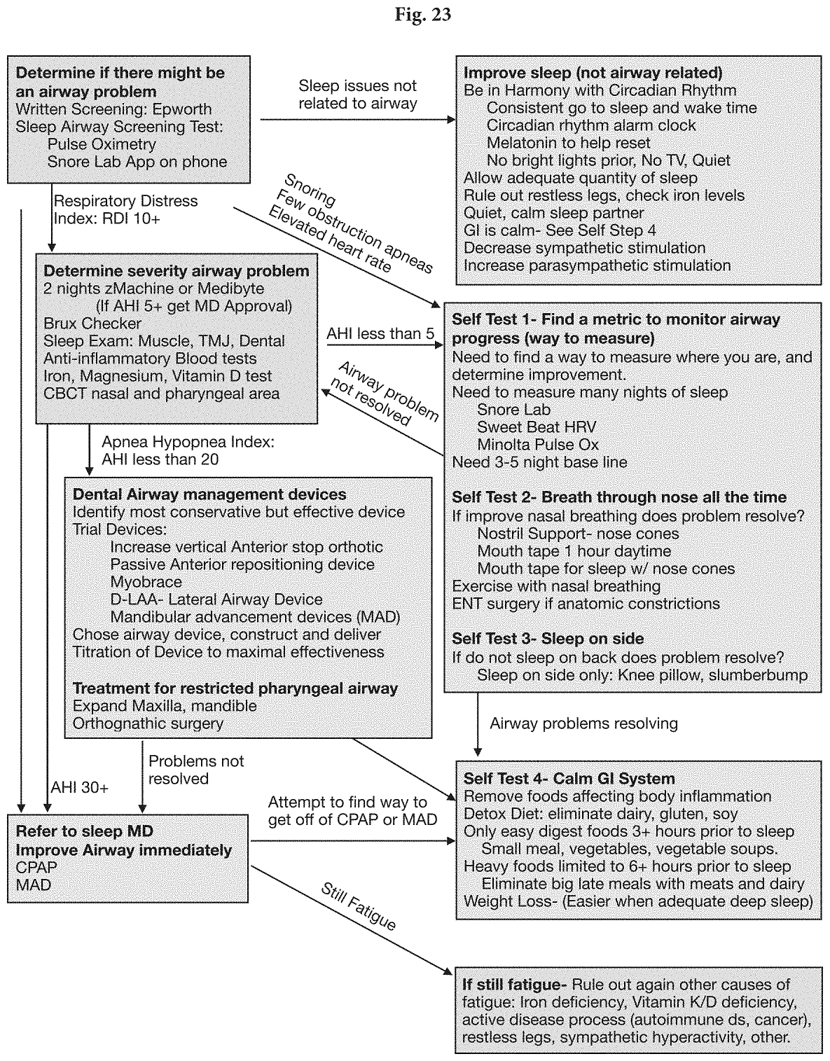

FIG. 23A is a schematic cross-sectional representation of a patient's jaw in a natural state. In the case of FIG. 23A, the right condyle stays in fossa. and separation space between the upper and lower appliances 230, 260.

FIG. 23B is a schematic cross-sectional representation of a patient's jaw in a displaced state. In the case of FIG. 23B, the left condyle is moved forward out of fossa by the mandibular advancement device, moving the jaw to the right.

The normal bite (occlusal) position for the teeth is shown, and particularly the relationship between the upper jaw and the lower jaw. In performing mandibular advancement treatment, it is desired to advance a lower jaw to a position relative to the upper jaw. The degree of advancement can depend upon clinical requirements. The relative displacement of the hinge point can be seen to have both horizontal and vertical components. Advancement of the lower jaw carries the tongue forward so that (particularly in sleep) there is a greatly reduced tendency for the tongue to impinge on the pharynx. The degree of advancement can be from the reflex or habitual closing path to the anterior border path.

Device 10 includes an upper appliance 12 and a lower appliance 14 which are fitted onto the upper and lower jaws. Appliance 12, 14 may be made from any material suitable for dental uses, such as a hard acrylic material, or methylmethacrylate or a polycarbonate resin thermoplastic. Such materials are known to those familiar with dental mouthpieces, and other materials may be used without departing from the intended scope herein.

Appliances 12 and 14 fit at least some of a user's upper and lower arches of teeth 13, 15, respectively. Extending upwardly from lower appliance 14 is a post or “fang” 16. Post 16 is shown as square-shaped, but other configurations are contemplated by the disclosure. Post 16, when in use, makes contact with a flat mating surface 18 of upper appliance 12. This contact between post 16 and surface 18 causes lower appliance 14, and consequently a user's lower jaw, to extend slightly sideways (laterally). Sideways extension of the lower jaw allows the air passage of the user to remain open, thereby preventing or minimizing snoring and improving breathing during sleep.

Post 16 may have a wide range of shapes, including various lengths, depths, and widths, to perform this function. The term “post” or “fang” is used to describe any such structure. Furthermore, post 16 may directly contact the upper appliance 12, or it may contact an attachment to upper appliance 12, such as a tab, an adjustable tab, any attachment extending upward, or any other attachment to upper appliance 12 which forms the flat surface 18.

The post 16 preferably has a removable and adjustable shim 20 which is inserted into an opening 22 found in the fang. The shim is shown as T-shaped, but can have other configurations without departing from the scope of the disclosure. The shim thickness is adjustable so as to allow adjustment of the amount of sideways (lateral) extension of the user's lower jaw. The shim may be made of the same material as the appliance 14, or any suitable dental material.

The upper appliance 12 has flat protruding surface 18 which engages the post 16. Surface 18 may be integral to the appliance 12 or it may be formed by a separate tab or post. Post or fang 16 can slide along the surface 18 which in turn moves the fang and the lower appliance and lower jaw sideways.

In accordance with another aspect of the disclosure, an air supply tube or connector 30, 32 is provided to upper and lower appliances 12, 14, respectively, to provide continuous or intermittent supply of air into the patient's mouth. The post or fang 16 has a gap 34 formed between parallel walls 36, 38 for accommodating the air supply connector 32 therebetween.

Airway tubes 40, 42 are formed on opposite right and left sides of the upper and lower appliances 12, 14 which extend around the perimeter of both sides of the upper and lower appliances to accommodate an air supply which passes through opening 44.

A channel 13 is formed in upper appliance 12 or lower appliance 14 via walls 15, 17 to accommodate deformable material or the user's teeth therein.

The air supply connector connects to an air supply such as a BPAP (Biaural Positive Airway Pressure) or PAP (Positive Airway Pressure) machine (not shown) and the air supply tube 40, 42 carries high pressure air to the oropharynx or the back of the throat. The air can be intermittently supplied and may be activated when the patient takes a breath. The airway channels can be custom fitted or airway advanced.

Some existing appliances are used with a CPAP machine, which has the disadvantage of a constant air supply being provided, which may not be comfortable or desirable for the patient.

In accordance with another embodiment of the disclosure, a temporary appliance 50 is provided. The appliance 50 has a similar appearance as the upper and lower appliances.

An acrylic or plastic tray 52 having a lower end 53 and two parallel side walls 53, 57 is used to hold a liner or a dental impression or deformable material 54, which is used for an individualized or customized fit. An advantage of this device is that it is low cost and is easily adjustable to each patient. The appliance can also be made virtually next to the patient's chair as they wait.

Deformable material 54 is bonded to the inner area 59 of the tray 52 and used for custom forming of a mold of the user's teeth for proper fitting during use. By using deformable material, each user can customize his or her anti-snoring device without the expense associated with having a dental mold prepared by a dental professional.

A suitable material for deformable material 54 is an ethylene-vinyl acetate copolymer resin. Any other suitable deformable materials may also be used. Typically, with such material, the material is heated to a temperature of about 150 Fahrenheit, through a microwave oven or by heating in hot water, for example, so as to place the material in its deformable state. A user then inserts the appliance and bites down, thereby deforming the material into the shape of the user's upper arch of teeth. The appliance is then removed and allowed to cool, thereby setting the material into a mold of the user's upper arch.

Likewise, lower appliance includes a tray filled with a deformable material. A mold of the lower arch of teeth is formed in connection with upper arch.

In accordance with another embodiment of the disclosure, a custom fit appliance tray 60 made of acrylic or plastic material is made directly on a model of the patient's upper and lower jaws. Again, the tray 60 has a lower portion 61 and two parallel side walls 63, 65 would have a similar appearance as the appliances.

In accordance with another embodiment of the disclosure, an appliance 70 is used for a sports application. The appliance has a plastic or acrylic shell 72, and a boil or bite impression material 74 therein between lower portions 75 and two parallel side walls 77, 79. During sports performance, such as in running or jogging, airway resistance may occur. The appliance helps open the airway at least on a temporary basis to increase airway performance.

In accordance with another embodiment of the disclosure, a viscoelastic energy absorbing material 80 is supplied on each side of the patient's teeth to have absorptive properties and anti-concussive properties by providing a gap in the patient's jaws. The absorbing material 80 can be used in conjunction with any of the appliances described herein.

An alternate embodiment of the disclosure is shown. Upper appliance body 100 has a U-shaped channel 101 formed in a central portion 103 thereof. Central portion 103 has a thinner cross section than outer lateral portions 105, 107 to facilitate placement of moldable material on an upper portion of the appliance or the user's teeth and has a pair of adjustable wings or engaging members 102, 104 which are slidably attached to opposed walls 109, 111 of upper appliance 100. Wings 102, 104 have an angled front wall 106, having an acute angle of about 30 degrees or 45 degrees from horizontal and a substantially straight rear wall 108. Several screws or other fasteners 110 are threaded through holes 112, 114 in side walls 115 of the wings. A pair of elongated lateral grooves or channels 116, 118 is formed in opposed side walls 119 of the upper appliance 100. Wings 102, 104 can slide laterally along channels 116, 118 and is secured to the preferred position via screws 110 which extend into channels 116, 118.

Added material or shim 120 is placed or mounted at an underside of the appliance body 100 at a rear or posterior portion of the body to provide more of a vertical gap between the appliances and to allow the tongue to more easily move forwardly. Preferably the shim has a height of about 2 millimeters (0.0002). However, other thicknesses are contemplated by the disclosure.

The lower appliance has a body 121 having a substantially U-shaped channel 122 formed in a central portion 134 of the body. A pair of laterally opposed wings or protrusions 124, 126 are permanently or fixedly attached to opposite side walls 128, 130 of body 121. Alternatively, the wings can be integrally formed as part of the body 121 and are contiguous with body 121. The wings 124, 126 each has an angled rear wall 130 (having an obtuse angle of about 150 degrees or 135 degrees from horizontal) which engages the angled front wall 106 of the movable upper wings 102, 104. An additional amount of solid material or shim 132 is added or stacked onto an upper rear portion of body 121 to facilitate movement of the tongue forwardly. The stack or shim can be about 2 millimeters in height or so on the body for the back teeth to enable the tongue to easily slide forward.

Central portion 134 of the body has a thinner cross section than the outer portions 136, 138 to facilitate placement of moldable material. There is also a notch 140 formed in the central area to receive and hold a measurement device (not shown).

Air channels can be incorporated in temporary or permanent appliances to provide room air or oxygen to the user. The hose or tube can be easily attached or removed.

Another advantage of the appliance is that it can be temporary or custom.

The exemplary embodiment has been described with reference to the preferred embodiments. Obviously, modifications and alterations will occur to others upon reading and understanding the preceding detailed description. It is intended that the exemplary embodiment and the appended claims be construed as including all such modifications and alterations.

Next, an exemplary method of patient use and implementation will be described.

A first step includes patient assessment. Conduct complete patient medical history, which may include the following questions: Does the patient snore? Do the patient often feel tired, fatigued, or sleepy? Has anyone observed the patient stop breathing during sleep? Has the patient been treated for high blood pressure?

If a patient answers “YES” to two or more questions, then the patient may be at risk for OSA.

An embodiment of the device can use different lengths of elastic straps to gradually and incrementally titrate (i.e. advance) the mandible laterally/to the side. Different strengths of elastic straps match the strength of pull to the musculature of the patient.

An embodiment of the device allows freedom of lateral mandibular movement. Air flow through the oral pharynx is increased either through the advancement of the mandible, increased vertical opening, or both, while thermoformed custom trays limit tooth movement.

A next step includes Impressions & Bite Registration. Take upper and lower dental impressions. The extension of the impressions should go to the height of contour of the gingiva on all sides of the teeth. Take a wax bite with the patient in an unprotruded centric occlusion with the bite open 8 to 10 mm in the anterior region. Send models and wax bite to an authorized dental laboratory.

A next step includes Delivery of appliance to patient. Fit the upper and lower appliances without the elastic straps to check for comfort and to make sure there is no gingival impingement. The may feel snug to the patient for the first five to ten minutes. Remove upper and lower appliance.

Wet two elastic straps of the same color and length, then attach each to the lower appliance, rotating the strap on the button hook to seat. The logo on the strap should be facing the appliance. Repeat procedure to attach straps to upper appliance.

Seat the upper appliance first and have the patient move the mandible forward while pushing down on the anterior portion of the lower appliance until it snaps into place. Question the patient for comfort of the appliance on the teeth as well as TMJ comfort.

Check the posterior bite pads for even occlusion. If either side is high, conservatively grind the high side until both sides occlude evenly in the protruded position with straps in place.

To avoid unnecessary office visits, you may send extra straps with the patient to use in advancing the mandible further or to replace stretched out straps. Instruct the patient to chew on something every morning after wearing the appliance to help return condyles to normal position.

A next step includes Patient Follow Up. Instruct your patient to call the office the day after the first night of wearing the appliance. Side effects may include clinching, sore anterior teeth, TMJ sensitivity and increased saliva flow; these symptoms should abate significantly or disappear completely within ten days. If mild TMJ discomfort is reported, replace existing straps on the appliance with softer or longer straps. (e.g. replace #1 yellow with #1 white).

If the patient has discomfort in one TMJ, adjust the bite pads as necessary (pain usually is on the side where the pad is high). If the patient experiences pain in both TMJs, he or she should discontinue wearing the appliance and contact your office.

If after wearing the appliance one or two teeth are sore, it may be necessary to relieve the interior problem areas with an acrylic bur. A next step includes Titration (Advancement) & Vertical if an elastic strap is ⅛ of an inch longer than a new strap or the hole becomes oval, then it should be replaced with a new strap of the same color and length.

If the patient reports that there has been no significant lessening of apneic episodes or snoring, AND if the patient feels the mandible could be moved further forward without TMJ pain, then this may be addressed in one of three ways: 1) Replace the present straps with new straps of the same color that are one size shorter (e.g. replace #1 yellow with #2 yellow) 2) Replace the present straps with new, firmer straps of a different color that are one size longer (e.g. replace #2 yellow with #1 blue). 3) Increase vertical opening by adding up to 3 or 4 mm of acrylic or light cure composite to the occlusal surfaces of the mandibular bite pads.

The LAAD aims to create a widening of the upper airway configuration by posturing the mandible Laterally. Much less force needed for this movement. One condyle stays in the Fossa. A patient can alternate nights on which condyle is out of the fossa. The mandible is thus not held rigid, but can is free to move in this lateral position. Since the jaw is supported laterally, this can stop sleep grinding of teeth.

Sleep Airway Treatment Process

Some embodiments are directed toward a treatment process for the airway issues described above. A major problem that affects testing for SDB is that there are many variables that occur in the day that affects how well one breathes that night. Daily variables affecting SDB include: Foods eaten that are inflammatory, timing and size of last meal prior to sleep, emotional stress, medications taken, and nutritional deficiencies. Other variables affecting SDB include: Pain, inconsistent sleep and wake times, quality of prior nights sleep, sleep deficit, sleep room environmental noise and light, hour of lights out, and sleep partner moving or snoring. Multiple nights of testing (as opposed to a single night) are needed to identify a true pattern for anyone with SDB due to the afore mentioned variables.

The process starts with screening to determine if an airway problem might exist. Written screening exams for SDB include Epworth sleepiness scale, Pediatric sleep questionnaire, STOP-Bang Questionnaire, and the Berlin Questionnaire. Review of medical history for conditions that are related to SDB and OSA include: High blood pressure, gastroesophageal reflux disease, attention deficit disorder, and fatigue. Sleep airway screening tests include pulse oximetry (Konica Minolta Pulsox-300i Pulse Oximeter, Nonin WristOx2 3150 USB, Viatom Checkme O2 pulse oximeter), Snore Lab phone app for snoring detection, and heart rate variability testing (Sweetbeat HRV by Sweet Water Health with Polar H10 Heart Rate monitor). Screening oximetry should summarize the number of 2%, 3% and 4% desaturation to identify the patients at risk for having SDB. Current most oximetry analyzing software is only looking at 4% or greater desaturation, but can be modified to provide the number of 2% and 3% desaturations as the data is already there. This will help identify SDB patients in the earlier stages.

Patients at risk for SDB need further evaluation to determine the presence and severity of the airway problem. SDB can be verified with home sleep testing with a level 3 device for two or more nights (zMachine by General Sleep or Medibyte by Braebon). If the AHI is 5 or greater, medical approval from a qualified MD is needed to proceed with any therapies. Anyone with medical conditions such as high blood pressure or gastroesophageal reflux disease should be seen by a qualified medical sleep doctor. A polysomnogram level 1 sleep study may be ordered by the sleep physician for further validation of the severity of the SDB. A diagnosis by the sleep physician can then be made of pre-OSA (Upper Airway Resistance), mild OSA, moderate OSA, or severe OSA.

Severe OSA and moderate/severe OSA patients are best managed with current protocols and therapies for OSA including CPAP and or mandibular advance devices (MAD).

For the pre-OSA (Upper Airway Resistance) mild OSA and mild/moderate OSA patients a more conservative approach is warranted. Once the presence of a less severe SDB problem is determined, testing is done to determine which of the numerous causes of SDB a patient has. A systematic algorithm of testing is used to determine the specific factors for this patient. Much of the testing can be done at home by the patient and monitored by the patient. This self testing reduces costs and involves the patient in their disease and subsequent resolution. The health care provider oversees the data gathered and defines the next steps. Many nights of data can be gathered with many different trials to help sort through all the variables that affect SDB.

Prior to the start of self testing for SDB airway variables, dental and medical health status needs to be determined. A thorough dental exam is performed including TMJ exam, TMJ muscle exam, dental exam, periodontal exam. Sleep bruxing is assessed utilizing Brux Checker Red Mylar Orthotic (GreatLakes Orthodontics). A CT or a Conebeam CT is taken and evaluated for TMJ health and for an unobstructed path from the nostrils to the lung. A thorough medical exam is performed including, blood pressure, blood tests for inflammation, iron, magnesium, and vitamin D.

Sleep hygiene needs to be discussed and implemented with the patient. Improvement of sleep can be achieved by controlling variables that are not airway related. Sleep can be improved by: Being in harmony with the circadian rhythm, consistent go to sleep and wake time, circadian rhythm alarm clock, melatonin supplementation, no bright lights prior to sleep, no TV in bedroom, quiet room, adequate quantity of sleep, calm and quiet sleep partner, calm GI system, decrease sympathetic stimulation, increase parasympathetic stimulation.

For self testing to be effective a metric is needed that can monitor progress or lack there of. Unfortunately there is not one metric that can be used for every patient. The simpler the better as the more involved the self test the less likely a patient will follow through with the copious amount of testing and data gathering that is needed to sort through all the variables. Having the health care provider have access to the data at the same time as the patient is ideal. Self testing devices include: Snore Lab app with phone, Sweetbeat HRV app with phone and Polar H10 Heart Rate monitor, Konica Minolta Pulsox-300i Pulse Oximeter, and Viatom Checkme O2 pulse oximeter with iphone app. A base line needs to be determined consisting of 3 to 5 nights of testing.

Proper respiration when awake and asleep is through the nose. Conditions that interfere with nasal breathing include deviated septum, swollen nasal tissue, enlarged adenoids, and enlarged tonsils. Mouth breathing is especially detrimental to children as the tongue is not pressed against the maxilla which limits the growth of the maxilla further limiting the size of the nasal passages. Identify and correcting mouth breathing in children and adults is the first line of treatment for all SDB patients who are “pre” OSA.

Variables that can be altered to determine their effect on sleep airway include: Nostril Support (Mute Nasal Dilator by Rhinomed, Breath Right Nasal Strips), Mouth taping, Timing and size of the evening meal, removal of inflammatory foods from the diet (gluten, diary, soy), alcohol consumption, parasympathetic relaxation prior to sleep, and sleep on side only. If any of these correct the problem, no further testing is needed and the solution has been identified. Verify resolution of the SDB by repeating the type 3 sleep test originally used to diagnose the SDB.

For the pre-OSA (Upper Airway Resistance), mild OSA, or mild/moderate OSA patients who did not get resolution with the above trial therapies, a sleep airway assisting device may be effective. The Lateral Airway Assistance Device (LAAD) which moves the jaw laterally is ideal for these patients as it is more conservative than either a MAD which moves the jaw forward or CPAP. Many pre-OSA (Upper Airway Resistance) patients do not tolerate a MAD due to an increase in the TMJ pain and muscle pain they already have. The LAAD has less side effects than a MAD and is better tolerated by these pre-OSA (Upper Airway Resistance) patients.

A time consuming problem for both LAAD and MAD is titrating these devices so they are the most effective by altering the amount of vertical opening and the amount of mandibular displacement. Self testing (using the chosen monitor) can be run with trial versions of the D-LAA with different verticals and lateral advancements. The final LAAD is also titrated using the self testing method. Once the most effective position is determined, resolution of the SDB is verified by repeating the type 3 or type 1 sleep test originally used to diagnose the SDB.

While certain embodiments of the invention are described above, and FIGS. 1-22 disclose the best mode for practicing the various inventive aspects, it should be understood that the invention can be embodied and configured in many different ways without departing from the spirit and scope of the invention.

What is claimed is:

1. A guided airway device for insertion into a user's mouth that includes an upper row of teeth and lower row of teeth, each of the upper and lower rows of teeth including front teeth, the guided airway device comprising:
a lower appliance that generally conforms to and at least partially covers the user's lower row of teeth, the lower appliance having two ends such that an approximate center point is disposed adjacent the user's front teeth;
an upper appliance that generally conforms to and at least partially covers the user's upper row of teeth, the upper appliance having two ends such that an approximate center point is disposed adjacent the user's front teeth; and
a guide that laterally biases the upper and lower appliances so as to laterally separate the user's upper front teeth from the user's lower front teeth.

2. The guided airway device of claim 1, wherein the guide includes at least one ramp formed on at least one of the upper appliance and the lower appliance.

3. The guided airway device of claim 2, wherein the at least one ramp is formed at a side of the at least one of the upper appliance and the lower appliance spaced from the user's front teeth.

4. The guided airway device of claim 3, wherein the at least one ramp is formed to contact an opposing one of the upper appliance and the lower appliance at a corresponding side thereof.

5. The guided airway device of claim 2, wherein the guide includes at least one band connected to the upper appliance and the lower appliance at corresponding sides thereof.

6. The guided airway device of claim 2, wherein the at least one band is connected to the upper appliance and the lower appliance at respective positions thereon that are offset from each other.

7. The guided airway device of claim 1, wherein the guide includes at least one band connected to the upper appliance and the lower appliance at corresponding sides thereof.

8. The guided airway device of claim 7, wherein each of the upper appliance and the lower appliance includes a hook formed thereon, the at least one band configured to connect to the hook of each of the upper appliance and the lower appliance.

9. The guided airway device of claim 8, wherein the hooks of each of the upper appliance and the lower appliance are offset from each other.

10. The guided airway device of claim 1, wherein the guide includes at least one rod connected to the upper appliance and the lower appliance at corresponding sides thereof.

11. The guided airway device of claim 10, wherein the at least one rod is adjustable to adjust a distance of lateral separation between the upper appliance and the lower appliance.

12. The guided airway device of claim 10, wherein the guide includes at least one band connected to the upper appliance and the lower appliance at corresponding sides thereof.

13. The guided airway device of claim 12, wherein the at least one band is connected to at least one of the upper appliance and the lower appliance at a different position from the at least one rod.

14. The guided airway device of claim 1, further comprising a post formed on at least one of the upper and lower appliances that vertically separates the upper and lower appliances.

15. The guided airway device of claim 14, wherein the post is formed at the user's front teeth.

16. A guided airway appliance for insertion into a user's mouth that includes an upper row of teeth and lower row of teeth, each of the upper and lower rows of teeth including front teeth, the guided airway appliance comprising:
a guard that generally conforms to and at least partially covers one of the user's upper row of teeth and lower row of teeth, the appliance having two ends such that an approximate center point is disposed adjacent the user's front teeth; and
a guide that laterally biases the appliance so as to laterally separate the user's upper front teeth from the user's lower front teeth.

17. The guided airway appliance of claim 16, wherein the guide includes at least one ramp formed on the guard.

18. The guided airway appliance of claim 17, wherein the at least one ramp is formed at a side of the guard spaced from the user's front teeth.

19. The guided airway appliance of claim 18, wherein the at least one ramp is formed to contact an opposing one of the user's upper row of teeth and lower row of teeth at a corresponding side thereof.

20. A method of opening an airway of a patient having an upper row of teeth and lower row of teeth, each of the upper and lower rows of teeth including front teeth, the method comprising:
forming a lower appliance that generally conforms to and at least partially covers the patient's lower row of teeth, the lower appliance having two ends such that an approximate center point is disposed adjacent the patient's front teeth;
fitting the lower appliance to the patient's lower row of teeth;
forming an upper appliance that generally conforms to and at least partially covers the patient's upper row of teeth, the upper appliance having two ends such that an approximate center point is disposed adjacent the patient's front teeth;
fitting the upper appliance to the patient's upper row of teeth;
forming a guide on at least one of the upper and lower appliances; and
separating the patient's upper front teeth from the patient's lower front teeth laterally by biasing the upper and lower appliances apart with the guide.
